(19) United States
(12) Patent Application Publication
(10) Pub. No.: US 2025/0145660 A1
(43) Pub. Date: May 8, 2025

(54) SOLID PHASE PEPTIDE SYNTHESIS WASH PROCESS

(71) Applicant: Eli Lilly and Company, Indianapolis, IN (US)

(72) Inventor: Mark Richard BERGLUND, Zionsville, IN (US)

(21) Appl. No.: 18/837,488

(22) PCT Filed: Feb. 13, 2023
(86) PCT No.: PCT/US2023/012870

§ 371 Date: Aug. 9, 2024

Related U.S. Application Data
(60) Provisional application No. 63/310,335, filed on Feb. 15, 2022.

(51) Int. Cl. C07K 1/04

(52) U.S. Cl. CPC C07K 1/045

(57) ABSTRACT

A method is disclosed for washing an amino acid that has been added to a solid phase resin during solid phase peptide synthesis (SPPS), comprising:
adding a first quantity of wash solvent, wherein once the wash with the first quantity of wash solvent is complete, sending the first quantity of wash solvent to waste; and
adding a second quantity of wash solvent, wherein once the wash with the second quantity of wash solvent is complete, sending the second quantity of wash solvent to a container, wherein the second quantity of wash solvent will be used in the first wash for the next amino acid that is added.

### BACKGROUND

Solid Phase Peptide Synthesis (SPPS) is the method and system most commonly used to synthesize polypeptides and amino acid sequences. WO 2021/158444 describes SPPS (WO 2021/15844 is expressly incorporated herein by reference). As part of the SPPS sequence, the growing peptide-on-resin solid-phase is washed with solvent to remove residual reagents and byproducts in order to prepare the solid-phase for the next amino acid addition. The wash solvent is then commonly deemed a waste stream and discarded. As this wash step is typically repeated upwards of 40 times during the synthesis of a polypeptide (or even more times for a long peptide) a great deal of solvent is used. Peptide drugs are now being produced in large scale batches using SPPS and these production methods use a great deal of solvent. Methods to reduce overall solvent usage will be of both economic and environmental benefit and are needed.

### SUMMARY

Disclosed herein is a method for washing an amino acid that has been added to a solid phase resin during solid phase peptide synthesis (SPPS). The method includes adding a first quantity of wash solvent, wherein once the wash with the first quantity of wash solvent is complete, sending the first quantity of wash solvent to waste; and adding a second quantity of wash solvent, wherein once the wash with the second quantity of wash solvent is complete, sending the second quantity of wash solvent to a container. The second quantity of wash solvent will be used in the first wash for the next amino acid that is added.

Also disclosed herein is a system for washing an amino acid that has been added to a solid phase resin during solid phase peptide synthesis (SPPS). The system includes a first container and a waste receptacle; and a first quantity of wash solvent and a second quantity of wash solvent. Once a wash with the first quantity of wash solvent is complete, the first quantity of wash solvent is sent to the waste receptacle. Once the wash with the second quantity of wash solvent is complete, the second quantity of wash solvent is sent to the first container. The second quantity of wash solvent will be used in the first wash for the next amino acid that is added.

### BRIEF DESCRIPTION OF THE FIGURES

FIG. 1 shows an SPPS System combined with a Wash System as described herein.

FIG. 2 shows a Wash System as described herein.

FIG. 3 shows the decrease in piperidine concentration across progressive wash streams (washes were stirred).

FIG. 4 shows concentration plotted against wash cycle number in Example 1 to generate a function to determine the piperidine concentration in the final wash at steady-state.

FIG. 5 shows piperidine concentration across progressive wash streams in Example 2 (washes not stirred).

FIG. 6 shows concentration plotted against wash cycle number in Example 2 to generate a function to determine the piperidine concentration in the final wash at steady-state.

### DESCRIPTION

The present invention recognizes that successive washes of an amino acid during SPPS provide cleaner wash solvent streams that still contain substantial wash value. Said another way, with each successive wash after an amino acid is added during SPPS, the wash solvent leaving the reactor has lower levels of residual reagents and byproducts than the initial reactor concentration. Even more specifically, the residual reagents in the waste streams are present at orders of magnitude less than they are in the SPPS reactor at the start of the wash cycle. By collecting the waste stream(s) from one amino acid cycle and using it (them) for the majority of the next amino acid wash cycle, the total solvent used in SPPS can be reduced dramatically.

For instance, after coupling an NH₂ protected amino acid (commonly Fmoc) to the polypeptide, the NH₂ protecting group (Fmoc) is normally removed via treatment with a weak base (commonly a secondary amine, specifically piperidine) in substantial molar excess. Upon completing the removal of the NH₂ protecting group, washing occurs to remove the excess base reagent and reaction byproducts. A common wash methodology consists of adding solvent to the SPPS reactors, stirring for a period of time to blend the contents, then draining the liquid phase to waste. This process is iteratively repeated until the residual base reagent and reaction byproducts are below a predefined threshold. The drained liquid phases are commonly treated as waste.

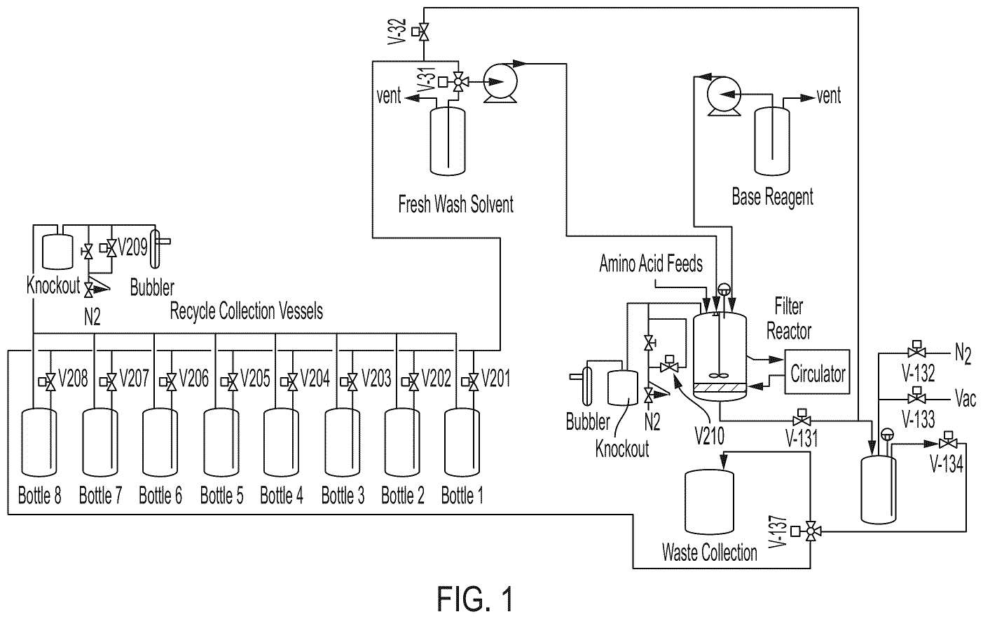

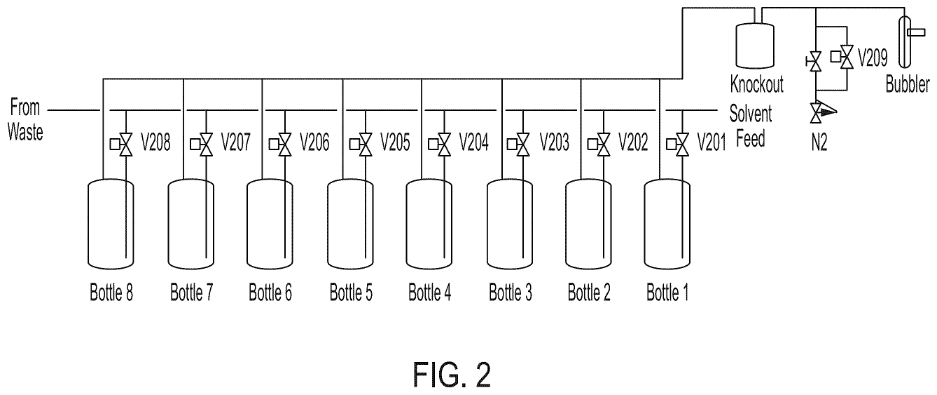

The different quantities of solvents that are used for each wash are generally the same solvent. However, in the present embodiments, the drained liquid phases are handled differently as shown in FIG. 1. The first wash is charged to the reactor, stirred, and drained to waste. The subsequent wash is charged to the reactor, stirred, then drained to a collection vessel (which is bottle 1). The third wash is charged, stirred, and drained to a second collection vessel (bottle 2). This is repeated until the wash cycle is completed. These collection vessels are retained until the NH₂ protecting group from the next amino acid residue has been removed and the vessel needs to be washed again. For this wash cycle, the first wash is charged from the first collection vessel from the previous wash cycle (e.g., from bottle 1), stirred, and drained to waste. The second wash is charged from the second collection vessel (from bottle 2), stirred, and drained to the first collection vessel (e.g., goes to bottle 1). The third wash is charged from the third collection vessel (from bottle 3), stirred, and drained to the second collection vessel (e.g., goes to bottle 2). This is repeated until the wash cycle is nearly complete. For the last wash, fresh wash solvent is charged to the reactor, stirred, and drained to the final collection bottle. This wash sequence is then repeated for all wash cycles following the NH₂ protecting group removal. A more detailed drawing of the wash equipment is given in FIG. 2.

Table 1 below shows how this process works, when there are three washes per amino acid addition. (Of course, this concept may be scaled, as appropriate, if more washes are desired for each amino acid addition reaction.) In the Table 1 below, “Wash Source” refers to the location where the solvent comes from for use in the washing step. “Wash Destination” refers to the location where the solvent is collected after the washing step. “Fresh” refers to a clean, new batch of solvent that has not been previously used in the washing process.

Similarly, this technology can be applied to different wash methodologies. Rather than stirred, the washes could be independent plug-flow washes, or a mix of stirred and plug-flow washes, where the collection and storage is analogous to the description above. Additionally, a single continuous plug flow wash effluent could be diverted into different holding tanks. For example, the first 10% of the effluent could be diverted to waste, the next 10% to collection vessel 1, so on and so forth. The next wash cycle could stack charges from these collection vessels to feed the next plug flow wash, with just the last 10% of the wash solvent consisting of fresh wash solvent.

The wash system is designed in such a way that it can be an add-on system that seamlessly plugs into an existing SPPS system. All that is needed is a tie-in where the existing fresh solvent system enters and another tie-in where the waste exits.

In addition to the embodiments outlined above, further embodiments may be designed that utilizes the fact that the first AA (amino acid) cycle washes are much cleaner than subsequent cycles as they start off as fresh solvent. In that case, it may be worth not sending any of the AA cycle 1 washes to waste and similarly not using fresh solvent for any washes on AA cycle 2. This saves 1 wash charge. In the case where this process is used to make a 10 peptide fragment, an example may be run that uses 8 fresh solvent washes for the addition of the first AA and then 1 fresh wash at the end of the wash cycle for each of the next 9 AA-thus resulting in a total of 17 washes. The strategy in table below reduces this to 16 washes, so in that example it saves 6% of the solvent. Likewise, in the case where we are making a tetramer (e.g., 4 amino acids) using the process of 6 fresh solvent washes for the first AA and one fresh solvent wash at the end of the wash cycle for each of the remaining 3 AAs, that would be a total of 9 washes—and the fact that you could save one wash using the method of the table below would mean that there would be only 8 washes (resulting in a saving of 11%). This strategy is shown below in Table 2.

TABLE 1
Example of current method using three washes

| AA | Wash 1 | Wash 1 | Wash 2 | Wash 2 | Wash 3 | Wash 3 | Wash n | Wash n |
|---|---|---|---|---|---|---|---|---|
| Cycle# | Source | Destination | Source | Destination | Source | Destination | Source | Destination |
| 1 | Fresh | Waste | Fresh | Bottle 1 | Fresh | Bottle 2 | Fresh | Bottle n − 1 |
| 2 | Bottle 1 | Waste | Bottle 2 | Bottle 1 | Bottle 3 | Bottle 2 | Fresh | Bottle n − 1 |
| 3+ | Bottle 1 | Waste | Bottle 2 | Bottle 1 | Bottle 3 | Bottle 2 | Fresh | Bottle n − 1 |

TABLE 2
Example of current method using n washes

| AA | Wash 1 | Wash 1 | Wash 2 | Wash 2 | Wash 3 | Wash 3 | Wash n | Wash n |
|---|---|---|---|---|---|---|---|---|
| Cycle# | Source | Destination | Source | Destination | Source | Destination | Source | Destination |
| 1 | Fresh | Bottle 1 | Fresh | Bottle 2 | Fresh | Bottle 3 | Fresh | Bottle n |
| 2 | Bottle 1 | Waste | Bottle 2 | Bottle 1 | Bottle 3 | Bottle 2 | Bottle n | Bottle n − 1 |
| 3+ | Bottle 1 | Waste | Bottle 2 | Bottle 1 | Bottle 3 | Bottle 2 | Fresh | Bottle n − 1 |

Table 2 uses the same definitions as Table 1 above. However, for purposes of comparison, the differences between Table 1 and Table 2 are shown in italics in Table 2.

Automation can control where each solvent charge originates as well as each solvent drain's destination.

Numerically, the extent of the solvent saving based on this process will depend on a multitude of factors, such as,
Adsorption to the solid-phase
Solvent holdup in the solid-phase (SPPS occurs on a resin bead, usually cross-linked polystyrene. The resin, along with the growing peptide, will swell with solvent. This is not free solvent and does not drain from the reactor, It does mix well with free solvent and the amount of this heldup solvent will impact the dilution effect of the wash)
Wash regime (stirred or plug-flow) and degree of channeling and dispersion in the case of plug-flow
Initial base content and final residual base target
# or amino acid residues. With the expectation that solvent will not be reused across batches, all vessels will be emptied at the end of the batch. This results in cleaner solvent being wasted. The more cycles, the greater the savings as this shutdown condition can be offset by a greater number of cycles.

With those clarifications here is the result of a simulation of the solvent savings with the following assumptions:
8 washes
Stirred washes
Equilibrium achieved
No solid-phase adsorption
Constant solvent holdup in the solid-phase for all cycles
Steady-state cycles (does not account for shutdown/startup)

The initial condition was 20 wt % (200,000 ppm) piperidine with a residual target of 500 ppm in the final wash stream. Assumed solvent holdup of 1,000 mL for the sake of simulation, though the result will not be impacted by scale. The simulation for the standard wash strategy is shown in Table 3, and Table 4 shows the results for the solid phase peptide synthesis wash strategy discussed previously. As shown, in this scenario the multi-stage wash strategy reduces the total solvent used from 8,920 mL (8 washes by 1,115 mL) to 1931 mL. This means the multi-stage wash system uses only 21.6% of the solvent used in the traditional wash, for any cycle after the first cycle. Alternatively, this is a 4.6× reduction in solvent use. The solvent savings are realized with no impact to the final state of the reactor as the same residual reagents and byproducts will be present at the same level upon completion of the wash step, regardless of the wash methodology used. This ensure that the wash methodology will not be linked to negative quality implications.

TABLE 3
Piperidine Removal with Standard Washing (all fresh solvent)

| | starting | | wash | wash solvent | | |
|---|---|---|---|---|---|---|
| | piperidine | Holdup | solvent | piperidine | | new |
| wash | concentration | volume | added | concentration | Drain | concentration |
| number | (ppm) | (mL) | (ml) | (ppm) | Volume | (ppm) |
| 1 | 200,000 | 1,000 | 1,115 | 0 | 1,115 | 94,563 |
| 2 | 94,563 | 1,000 | 1,115 | 0 | 1,115 | 44,710 |
| 3 | 44,710 | 1,000 | 1,115 | 0 | 1,115 | 21,140 |
| 4 | 21,140 | 1,000 | 1,115 | 0 | 1,115 | 9,995 |
| 5 | 9,995 | 1,000 | 1,115 | 0 | 1,115 | 4,726 |
| 6 | 4,726 | 1,000 | 1,115 | 0 | 1,115 | 2,234 |
| 7 | 2,234 | 1,000 | 1,115 | 0 | 1,115 | 1,056 |
| 8 | 1,056 | 1,000 | 1,115 | 0 | 1,115 | 500 |

TABLE 4
Piperidine Removal with Current Washing Method

| | starting | | wash | wash solvent | | |
|---|---|---|---|---|---|---|
| | piperidine | Holdup | solvent | piperidine | | new |
| wash | concentration | volume | added | concentration | Drain | concentration |
| number | (ppm) | (mL) | (ml) | (ppm) | Volume | (ppm) |
| 1 | 200,000 | 1,000 | 1,931 | 53,245 | 1,931 | 103,315 |
| 2 | 103,315 | 1,000 | 1,931 | 27,315 | 1,931 | 53,245 |
| 3 | 53,245 | 1,000 | 1,931 | 13,887 | 1,931 | 27,315 |
| 4 | 27,315 | 1,000 | 1,931 | 6,932 | 1,931 | 13,887 |
| 5 | 13,887 | 1,000 | 1,931 | 3,331 | 1,931 | 6,932 |
| 6 | 6,932 | 1,000 | 1,931 | 1,466 | 1,931 | 3,331 |
| 7 | 3,331 | 1,000 | 1,931 | 500 | 1,931 | 1,466 |
| 8 | 1,466 | 1,000 | 1,931 | 0 | 1,931 | 500 |

The present embodiments include a source for fresh wash solvent as well as one or more bottles (containers) for storing each successive wash. As used herein, containers, bottles, and vessels are intended to mean vessels that can contain fluid, e.g., a wash solvent. Specifically, as described above, there will be a waste collection vessel to which the first wash is drained—as this first wash is the dirtiest. There will be a first container for containing the second wash as well as separate containers for housing successive washes. The first wash will be sent to waste, whereas each subsequent wash will be sent to the respective containers. Thus, the third wash solvent will be sent to the second container, the fourth wash to the third container, and so on. When the ‘next’ AA is added, the first wash will come from the first container (and will be the solvent used in the ‘second wash’ of the previous step), the second wash will come from the second container (and will be the solvent used in the ‘third wash’ of the previous step) and so on. These washes will be recycled again into the containers and used again (as outlined in Table 1 (or in the embodiment of Table 2)).

The present embodiments may include a method for washing an amino acid that has been added to a solid phase resin, comprising:
adding a first quantity of wash solvent, wherein once the wash with the first quantity of wash solvent is complete, sending the first quantity of wash solvent to waste; and
adding a second quantity of wash solvent, wherein once the wash with the second quantity of wash solvent is complete, sending the second quantity of wash solvent to a container, wherein the second quantity of wash solvent will be used in the first wash for the next amino acid that is added.

In some embodiments, this method may further comprise a third quantity of wash solvent, wherein once the wash with the third quantity of wash solvent is complete, sending the third quantity of wash solvent to a container, wherein the third quantity of wash solvent will be used in the second wash for the next amino acid that is added.

This process may repeat iteratively, using successive containers and quantities of solvent being re-used during the next amino acid addition, until the last wash is done with fresh solvent.

The present embodiments may include a system for washing an amino acid that has been added to a solid phase resin, comprising a first container and a waste receptacle; and a first quantity of wash solvent and a second quantity of wash solvent. In this system, once the wash with the first quantity of wash solvent is complete, the first quantity of wash solvent is sent to the waste receptacle, wherein, once the wash with the second quantity of wash solvent is complete, the second quantity of wash solvent is sent to the first container, and wherein the second quantity of wash solvent will be used in the first wash for the next amino acid that is added.

This system may further comprise a second container and a third quantity of wash solvent, wherein once the wash with the third quantity of wash solvent is complete, the third quantity of wash solvent is sent to the second container, wherein the third quantity of wash solvent will be used in the second wash for the next amino acid that is added.

The system may include additional containers and additional quantities of solvent that may be iteratively and repeatedly use, until that last wash is clean solvent.

The present embodiments also provide for a product (a peptide or AA sequence) made by the processes disclosed herein. Any wash solvent useful with SPPS may be used with the solvent recycling methods described herein. A few non-limiting examples of wash solvents useful with SPPS include N,N-dimethylformamide (DMF) and isopropyl acetate (IPAC). The methods described herein are able to reduce SPPS base levels (e,g, piperidine) to less than about 1500 ppm, less than about 1000 ppm, or less than about 500 ppm (i.e., levels needed to proceed with the next amino acid addition in the SPPS route). The methods described herein are able to reduce the volume of wash solvent used in SPPS by at least about 65%, by at least about 70%, by at least about 75%, by at least about 80%, or by at least about 85% compared to using only fresh wash solvent. The methods described herein are also able to reduce the volume by about 65%, by about 70%, by about 75%, by about 80%, or by about 85% compared to using only fresh wash solvent.

### EXAMPLES

### Example 1

A system was developed to perform the washes as claimed (FIG. 1). The system consisted of an SPPS reactor containing NH₂-Ser on Sieber resin. The solid was swelled with DMF and drained, then swelled with DMF and drained a second time. The experimental procedure consisted of charging nominally 400 mL of 18.6 wt. % (20.0 v/v %) piperidine in N,N-dimethylformamide (DMF) to the reactor, stirring for one minute, and draining to waste. A second charge, stir and drain were done. Then, nominally 400 mL of fresh DMF was charged to the reactor, stirred for 1 minute, and drained, collecting a sample of the drained solvent. The drained solvent was collected in Bottle 1. This was repeated seven times (a total of eight times). Each time the drained wash was sampled and collected in a successive bottle, per the process outlined in Table 2. These samples simulate the traditional wash strategy, using all fresh solvent.

After the eighth wash, two piperidine charge, stir, and drain cycles were completed to return the reactor piperidine concentration to where it was before the first wash cycle. This wash cycle followed the outline for Cycle 2 in Table 2, where all washes came from the collection bottles. The first drained wash went to waste, and the last seven were collected for Cycle 3. For this wash cycle, only the eighth wash was sampled, with the sample collected upon draining the reactor.

The reactor again underwent the two piperidine treatments. It was then washed eight times per the description for Cycle 3 in Table 2. The first seven washes came from the collection bottles, while the eight wash was fresh DMF. The eighth wash was sampled upon draining.

This process was repeated for 12 cycles, sampling the eighth wash upon draining. Additionally, samples were collected upon draining the second piperidine treatment for some cycles to confirm the reactor was starting at a consistent piperidine concentration each cycle.

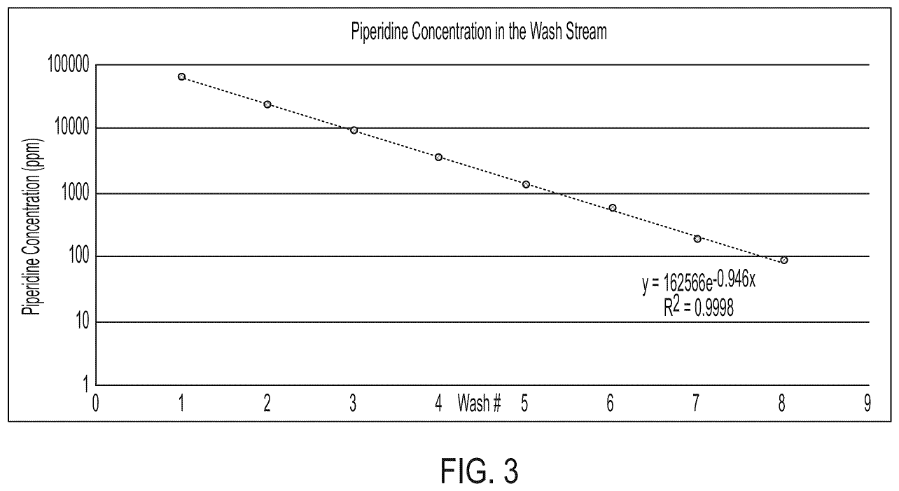

Upon completion of the experiment, the samples were analyzed by gas chromatography to quantify the piperidine concentration. First a curve was generated for the first wash cycle, showing the piperidine concentration as a function of the number of washes. This is shown in FIG. 3.

Samples were taken of piperidine drains to show consistency in starting concentration for each cycle, shown in Table 5.

TABLE 5
Consistency in starting concentration (Example 1)

| Cycle | Piperidine concentration (ppm) |
|---|---|
| 1 | 155981 |
| 2 | 154702 |
| 3 | Not taken |
| 4 | Taken, not analyzed |
| 5 | 158580 |
| 6 | Taken, not analyzed |
| 7 | 160446 |
| 8-12 | Not taken |

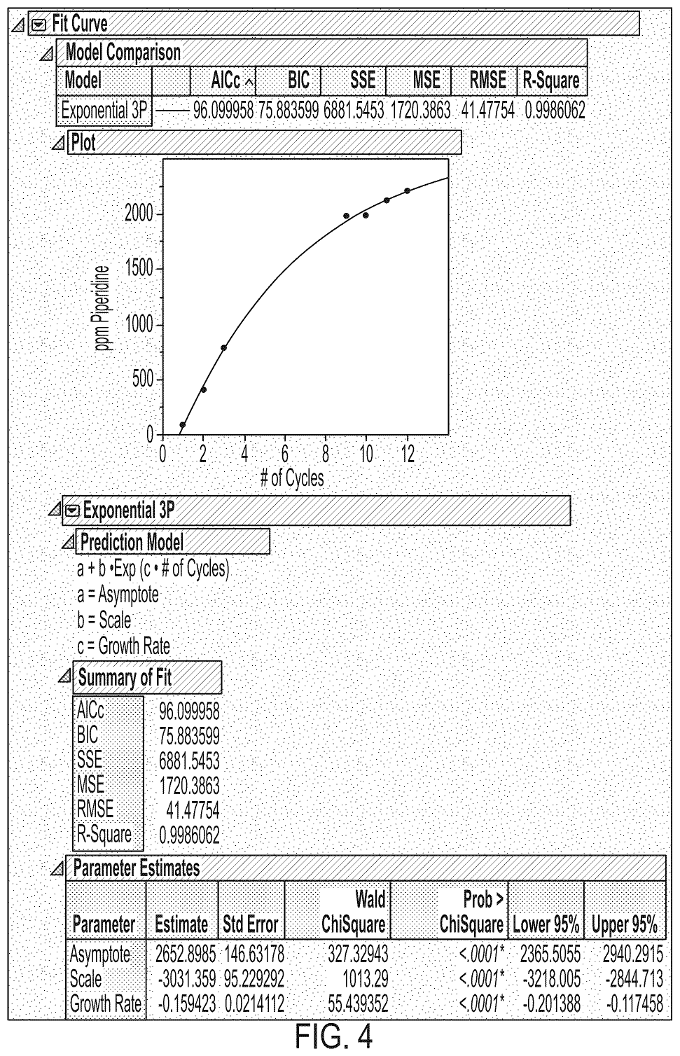

Then the final (eighth) wash concentration was plotted against the cycle number to generate a function to determine the piperidine concentration in the final wash at steady-state. Due to sampling issues, many of the early cycles did not return accurate results as residual piperidine sample in the sampling line inflated the results (first wash cycle was not impacted as all washes were sampled). After plotting the available data, statistical analysis software was used to determine the asymptote of the curve, which gives the expected equilibrium concentration. This is shown in FIG. 4

As can be seen, the steady-state piperidine concentration is estimated as 2653 ppm. This is equivalent to 4.4 fresh washes based on the regression of the curve in FIG. 3. As the wash volume was constant and the steady-state cycle having just one fresh wash, this proves that the solid phase peptide synthesis wash strategy described herein can save 77% of the solvent that would be used via the standard procedure (all washes to waste) for removing residual piperidine after removal of the amino acid protecting group. All results are shown in Table 6 and Table 7.

TABLE 6
Example 1 washing results

| | Piperidine | Piperidine | Fresh DMF charge | Piperidine concentration in | Final Wash |
|---|---|---|---|---|---|
| Cycle | Charge 1 (mL) | Charge 2 (mL) | for wash 8 (mL) | 2^{nd} drain (ppm) | concentration (ppm) |
| 1 | 402 | 403 | See Table 6 | 155981 | 92 |
| 2 | 403 | 398 | 0 | 154702 | 406 |
| 3 | 400 | 403 | 408^{#} | Not taken | 787 |
| 4 | 398 | 404 | 408 | Taken, not analyzed | Not taken |
| 5 | 404 | 402 | 405 | 158580 | Not taken |
| 6 | 408 | 403 | 410 | Taken, not analyzed | Not taken |
| 7 | 406 | 403 | 407 | 160446 | Not taken |
| 8 | 394 | 403 | 408 | Not taken | 3537* |
| 9 | 404 | 402 | 406 | Not taken | 1981 |
| 10 | 411 | 404 | 405 | Not taken | 1996 |
| 11 | 404 | 403 | 401 | Not taken | 2121 |
| 12 | 409 | 404 | 400 | Not taken | 2210 |

^{#}130 mL were accidentally charged during the 7^{th} wash. This should not have a major impact on the result for Cycle 2 and should have no effect on the final estimated steady-state result.
*This result appears to be an outlier and was not plotted in FIG. 4.

TABLE 7
Example 1 piperidine levels
Wash 1

| Wash # | Charge volume (mL) | Piperidine (ppm) |
|---|---|---|
| 1 | 385 | 65788 |
| 2 | 413 | 24540 |
| 3 | 403 | 9676 |
| 4 | 405 | 3446 |
| 5 | 405 | 1375 |
| 6 | 405 | 597 |
| 7 | 404 | 195 |
| 8 | 405 | 92 |

### Example 2

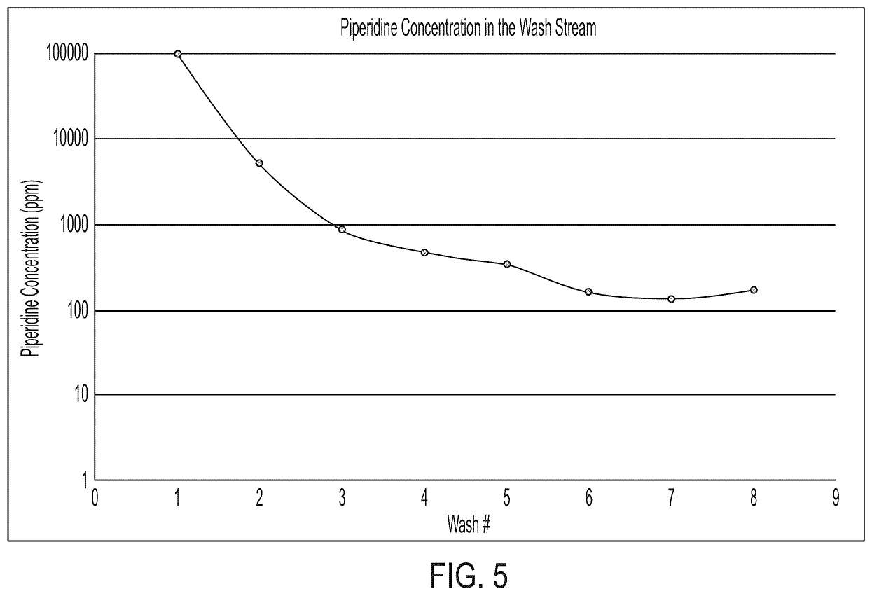

A similar experiment was conducted, only in this case washes were not stirred. The experiment consisted of charging piperidine, stirring, and draining twice, then charging nominally 400 mL of DMF. The DMF was then drained without stirring. For the first cycle, each wash was fresh solvent. Then two piperidine charges, stirs, and drains were completed and the wash repeated with only one fresh DMF charge, with the rest being sourced from the collection of washes from the previous cycle. The results for the first wash cycle are shown below. There is some variability in the later wash numbers likely owing to analytical and sampling variability at low levels and the non-ideality of the plug-flow washing as the bed distorts more and more each wash. This is shown in FIG. 5.

Samples were also taken of some piperidine drains to show consistency in starting concentration for each cycle, shown in Table 8. Due to an automation error in Cycle 4 in Table 8, the piperidine treatments in Cycle 4 were not stirred, resulting in a higher concentration.

TABLE 8
Consistency in starting material (Example 2)

| Cycle | Piperidine concentration (ppm) |
|---|---|
| 1 | 156446 |
| 2 | 155306 |
| 3 | Not taken |
| 4 | 179673 |
| 5 | Not taken |
| 6 | Not taken |
| 7 | 155919 |
| 8-10 | Not taken |

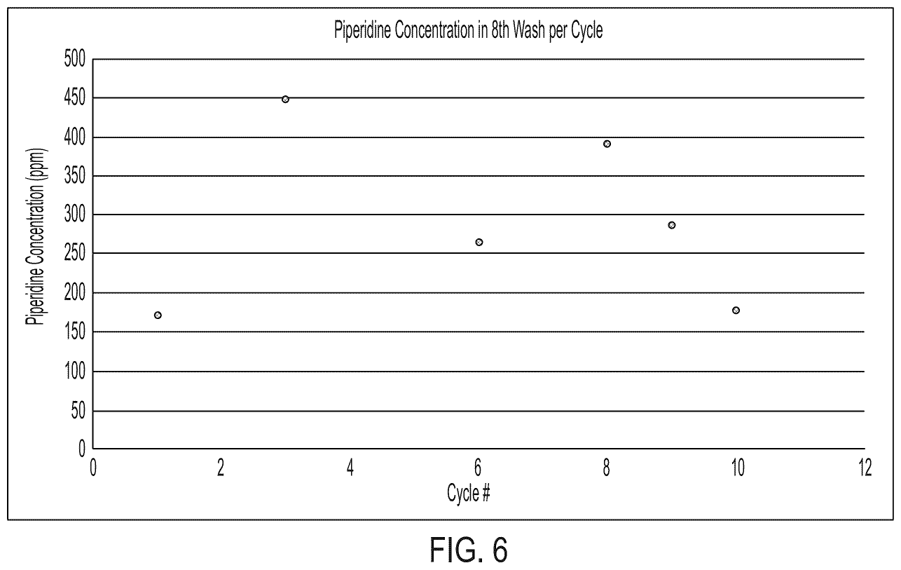

Then the final (eighth) wash concentration was plotted against the cycle number to generate a function to determine the piperidine concentration in the final wash at steady-state. Due to sampling issues, a few of the early cycles did not return accurate results as residual material from the piperidine sample left in the sampling port inflated the results (first wash cycle was not impacted as all washes were sampled). This is shown in FIG. 6.

As can be seen, the piperidine concentration is relatively variable across cycle number. This may be related to sample carry-over, but also related to how ideal the plug-flow was each cycle. The Cycle 10 result came from a sample of the bulk collection bottle, rather than the small sampling zone from which the rest of the samples were taken. This means that Cycle 10 is the truest result as the bulk bottle would not be subject to the sample port carry-over. The Cycle 10 result of 179 ppm is equivalent to or better than four fresh washes based on the curve in FIG. 3. As the wash volume was constant and the steady-state cycle having just one fresh wash, this proves that the solid phase peptide synthesis wash strategy described herein can save 75% or more of the solvent that would be used via the standard procedure (all washes to waste) that would likely be used with plug-flow washes for removing residual piperidine. As a double check of the effectiveness of the plug-flow washes, a stirred wash was conducted at the conclusion of Cycle 10. The result was 59 ppm piperidine, which proves that the plug-flow washes were effectively removing piperidine and not exiting the reactor with low piperidine concentration by failing to actual wash away the piperidine. All results are shown in Table 9 and Table 10.

TABLE 9
Example 2 washing results

| | Piperidine | Piperidine | Fresh DMF charge | Piperidine concentration | Final Wash |
|---|---|---|---|---|---|
| Cycle | Charge 1 (mL) | Charge 2 (mL) | for wash 8 (mL) | (ppm) | concentration (ppm) |
| 1 | 387 | 385 | See Table 6 | 156446 | 172 |
| 2 | 387 | 385 | 0 | 155306 | Not taken |
| 3 | 386 | 386 | 413 | Not taken | 448 |
| 4 | 387 | 387 | 410 | 179673^{#} | Not taken |
| 5 | 394 | 388 | 403 | Not taken | 879* |
| 6 | 398 | 395 | 404 | Not taken | 265 |
| 7 | 395 | 391 | 403 | 155919 | Not taken |
| 8 | 394 | 392 | 399 | Not taken | 391 |
| 9 | 395 | 385 | 403 | Not taken | 288 |
| 10 | 399 | 395 | 400 | Not taken | 179 |
| 10 stir | N/A | N/A | 405 | N/A | 59 |

^{#}Due to an automation error, the piperidine treatments in Cycle 4 were not stirred, resulting in a higher concentration.
*This result appears to be an outlier and was not plotted in FIG. 6.

TABLE 10
Example 2 piperidine levels
Wash 1

| Wash # | Charge volume (mL) | Piperidine (ppm) |
|---|---|---|
| 1 | 403 | 99326 |
| 2 | 404 | 5165 |
| 3 | 381 | 878 |
| 4 | 403 | 471 |
| 5 | 379 | 336 |
| 6 | 404 | 160 |
| 7 | 377 | 135 |
| 8 | 378 | 172 |

### Example 3

After Examples 1 and 2 demonstrated wash efficiency, a peptide was generated via SPPS using the new wash methodology. 60 mmol of 2-Chlorotrityl chloride (CTC) resin, 41.0 g, was charged to the SPPS reactor and a peptide containing nine amino acids was then synthesized following the standard SPPS cycle. The first cycle consisted of loading an amino acid on the resin with no Fmoc removal step, thus only 8 post-deprotection wash cycles needed to be completed. The standard synthesis called for the use of six DMF washes after deprotection consisting of 10 mL/g of resin. For this example, the target wash volume was 12 mL/g of resin with 8 wash stages. The washes were alternated between plug-flow (displacement wash) and stirred (slurry wash). The wash charge data used is shown in Tables 11, 12, and 13, where bold, italicized values represent the use of fresh DMF. Resin mass basis decreases each cycle due to sampling losses. In total, 6,360 g of DMF was used. Under the standard procedure, the total DMF use would have been 18,038 g. This is a 65% reduction. Starting with the third cycle onward, the solvent use was reduced by 80% compared to the standard procedure. Adding additional amino acids (and using additional wash cycles) to create longer peptides would drive the reduction percentage toward the 80% solvent saving level, as the higher fresh solvent use for the first cycle is outweighed by the more efficient recycled solvent usage in later cycles. Total mass balance is very closer to parity.

TABLE 11
Example 3 wash data

| | Resin | Resin | Wash1 (g) | | Wash2 (g) | | Wash3 (g) | | Wash4 (g) | |
|---|---|---|---|---|---|---|---|---|---|---|
| Cycle | (mmol) | (g) | Charge | Drain | Charge | Drain | Charge | Drain | Charge | Drain |
| Glu | 59.63 | 40.68 | 461 | 454 | 457 | 453 | 457 | 451 | 455 | 452 |
| Leu | 59.02 | 40.26 | 454 | 446 | 453 | 459 | 451 | 433 | 452 | 459 |
| Leu | 58.70 | 40.04 | 459 | 457 | 433 | 439 | 459 | 461 | 440 | 447 |
| Tyr | 58.39 | 39.83 | 439 | 441 | 461 | 466 | 447 | 450 | 446 | 454 |
| Glu | 58.08 | 39.62 | 466 | 470 | 450 | 456 | 454 | 461 | 455 | 461 |
| Ile | 57.80 | 39.43 | 456 | 457 | 461 | 468 | 461 | 466 | 486 | 491 |
| Phe | 57.52 | 39.24 | 468 | 470 | 466 | 470 | 491 | 498 | 454 | 462 |
| Ala | 57.22 | 39.03 | 470 | 467 | 498 | 498 | 462 | 468 | 456 | 456 |

TABLE 12
Example 3 wash data continued

| | Resin | Resin | Wash5 | | Wash6 | | Wash7 | | Wash8 | |
|---|---|---|---|---|---|---|---|---|---|---|
| Cycle | mmol | (g) | Charge | Drain | Charge | Drain | Charge | Drain | Charge | Drain |
| Glu | 59.63 | 40.68 | 456 | 448 | 456 | 454 | 456 | 455 | 456 | 453 |
| Leu | 59.02 | 40.26 | 448 | 440 | 454 | 450 | 455 | 449 | 453 | 464 |
| Leu | 58.70 | 40.04 | 450 | 446 | 449 | 457 | 464 | 476 | 456 | 438 |
| Tyr | 58.39 | 39.83 | 457 | 455 | 476 | 486 | 438 | 444 | 459 | 438 |
| Glu | 58.08 | 39.62 | 486 | 486 | 444 | 452 | 438 | 448 | 448 | 435 |
| Ile | 57.80 | 39.43 | 452 | 454 | 448 | 451 | 435 | 445 | 448 | 433 |
| Phe | 57.52 | 39.24 | 451 | 456 | 445 | 452 | 433 | 448 | 441 | 440 |
| Ala | 57.22 | 39.03 | 452 | 455 | 448 | 444 | 440 | 457 | 453 | 426 |

TABLE 13
Example 3 wash data totals

| | Resin | Resin | Total | Total |
|---|---|---|---|---|
| Cycle | mmol | (g) | Charge | Drain |
| Glu | 59.63 | 40.68 | 3655 | 3622 |
| Leu | 59.02 | 40.26 | 3622 | 3600 |
| Leu | 58.70 | 40.04 | 3611 | 3621 |
| Tyr | 58.39 | 39.83 | 3623 | 3634 |
| Glu | 58.08 | 39.62 | 3641 | 3670 |
| Ile | 57.80 | 39.43 | 3647 | 3665 |
| Phe | 57.52 | 39.24 | 3648 | 3696 |
| Ala | 57.22 | 39.03 | 3679 | 3671 |

Upon completion, the peptide was analyzed for quality using Ultra High Pressure Liquid Chromatography with UV detection (UHPLC-UV) (peak area percentage). The overall purity of the peptide was measured at 96.5%, which is on par with historical results. Additionally, the last two washes from the final cycle, wash 7 and 8 for Ala, were analyzed for residual piperidine. The results were 1,218 ppm for wash 7, and 501 ppm for wash 8, right at the standard 500 ppm target of the original process. In summary, a 9 mer peptide was synthesized using the solid phase peptide synthesis wash system described herein. The peptide quality was on par with historical data while reducing DMF use in the post-deprotection wash by 65%, while meeting the same residual piperidine level in the final wash.

### Example 4

The same 9 mer peptide from Example 3 was generated using the same method as Example 3 but using isopropyl acetate (IPAC) rather than DMF as the wash solvent. The peptide was again generated using the solid phase peptide synthesis wash system described herein. For the synthesis, the target wash volume was 12 mL/g of resin with 8 wash stages. The washes were alternated between plug-flow (displacement wash) and stirred (slurry wash). The wash charge data is given in Tables 14, 15, and 16, where the bold, italicized values represent the use of fresh IPAC. Resin mass basis decreases each cycle due to sampling losses. In total, 5,994 g of IPAC were used with the solid phase peptide synthesis wash strategy described herein. Under the standard procedure, the total IPAC use would have been 16,710 g. This is a 64% reduction. Starting with the third cycle onward, the solvent use was reduced by 79% from the standard procedure. Adding additional amino acids (and using additional wash cycles) to create longer peptides would drive the solvent reduction percentage toward the 80% solvent saving level, as the higher fresh solvent use for the first cycle is outweighed by the more efficient recycled solvent usage in later cycles. Total mass balance is not as close to parity as in Example 3. This can be explained by the reduction in resin swelling during IPAC washes (compared with the deprotection reaction carried out in DMF), which results in more solvent drained than charged in the wash step.

TABLE 14
Example 4 wash data

| | Resin | Resin | Wash1 (g) | | Wash2 (g) | | Wash3 (g) | | Wash4 (g) | |
|---|---|---|---|---|---|---|---|---|---|---|
| Cycle | mmol | (g) | Charge | Drain | Charge | Drain | Charge | Drain | Charge | Drain |
| Glu | 61.21 | 40.8 | 425 | 466 | 426 | 463 | 427 | 411 | 429 | 450 |
| Leu | 60.87 | 40.6 | 466 | 505 | 463 | 500 | 411 | 420 | 450 | 448 |
| Leu | 60.50 | 40.3 | 500 | 577 | 420 | 439 | 448 | 459 | 403 | 402 |
| Tyr | 60.15 | 40.1 | 439 | 527 | 459 | 508 | 402 | 409 | 423 | 428 |
| Glu | 59.81 | 39.9 | 508 | 571 | 409 | 431 | 428 | 437 | 397 | 400 |
| Ile | 59.49 | 39.7 | 431 | 442 | 437 | 453 | 400 | 411 | 453 | 453 |
| Phe | 59.21 | 39.5 | 453 | 458 | 411 | 431 | 453 | 464 | 431 | 434 |
| Ala | 58.87 | 39.3 | 431 | 437 | 464 | 458 | 434 | 436 | 441 | 444 |

TABLE 15
Example 4 wash data continued

| | Resin | Resin | Wash5 | | Wash6 | | Wash7 | | Wash8 | |
|---|---|---|---|---|---|---|---|---|---|---|
| Cycle | mmol | (g) | Charge | Drain | Charge | Drain | Charge | Drain | Charge | Drain |
| Glu | 59.63 | 40.68 | 427 | 409 | 426 | 439 | 426 | 407 | 426 | 441 |
| Leu | 59.02 | 40.26 | 409 | 403 | 439 | 426 | 407 | 405 | 441 | 441 |
| Leu | 58.70 | 40.04 | 426 | 423 | 405 | 399 | 441 | 461 | 432 | 422 |
| Tyr | 58.39 | 39.83 | 399 | 397 | 461 | 452 | 422 | 431 | 427 | 432 |
| Glu | 58.08 | 39.62 | 452 | 453 | 431 | 428 | 432 | 449 | 434 | 424 |
| Ile | 57.80 | 39.43 | 428 | 431 | 449 | 446 | 424 | 441 | 430 | 419 |
| Phe | 57.52 | 39.24 | 446 | 441 | 441 | 442 | 419 | 435 | 431 | 420 |
| Ala | 57.22 | 39.03 | 442 | 438 | 435 | 434 | 420 | 435 | 429 | 420 |

TABLE 16
Example 4 wash data totals

| | Resin | Resin | Total | Total |
|---|---|---|---|---|
| Cycle | mmol | (g) | Charge | Drain |
| Glu | 61.21 | 40.8 | 3412 | 3486 |
| Leu | 60.87 | 40.6 | 3486 | 3548 |
| Leu | 60.50 | 40.3 | 3475 | 3581 |
| Tyr | 60.15 | 40.1 | 3431 | 3585 |
| Glu | 59.81 | 39.9 | 3491 | 3592 |
| Ile | 59.49 | 39.7 | 3451 | 3498 |
| Phe | 59.21 | 39.5 | 3486 | 3524 |
| Ala | 58.87 | 39.3 | 3495 | 3501 |

Upon completion, the peptide was analyzed for quality using UHPLC-UV (peak area percentage). The overall purity of the peptide measured at 95.0%. Additionally, the last two washes from the final cycle, wash 7 and 8 for Ala21, were analyzed for residual piperidine. The results were 1,151 ppm for wash 7, and 240 ppm for wash 8, well below the 500 ppm target of the original process. This was anticipated as the resin swells less in IPAC than DMF and less solvent retained in the resin will improve the wash efficiency of the system. In summary, a 9 mer peptide was synthesized using the solid phase peptide synthesis wash system described herein with IPAC as the wash solvent. The peptide quality was on par with historical data while reducing IPAC use in the post-deprotection wash by 64%, while surpassing the target residual piperidine level in the final wash.

### Example 5

A peptide was generated via SPPS using the new wash methodology with IPAC as the wash solvent. 100 mmol of CTC resin, 67.0 g, was charged to the SPPS reactor. A peptide consisting of sixteen amino acids was then synthesized following the standard SPPS cycle. The first cycle consists of loading amino acid on the resin and no Fmoc removal step, thus only 15 post-deprotection wash cycles needed to be completed. The standard synthesis called for an average of 9 DMF washes after deprotection consisting of 10 mL/g of resin. For the demonstration of the solid phase peptide synthesis wash system described herein, the target wash volume was 12 mL/g of resin with 8 wash stages, but was increased to 14 mL/g after the peptide mass grew with a major mass addition after the third cycle. The washes were alternated between plug-flow (displacement wash) and stirred (slurry wash). The wash charge data is given in Tables 17, 18, and 19, where the bold, italicized values represent the use of fresh IPAC. Resin mass basis decreases each cycle due to sampling losses. In total, 14,284 g of IPAC were used with the solid phase peptide synthesis wash strategy described herein. Under the standard procedure, the total IPAC use would have been 71,795 g. This is an 80% reduction. Starting with the third cycle onward, the solvent use was reduced by 86% from the standard procedure. Adding additional amino acids (and using additional wash cycles) to create longer peptides would drive the solvent reduction percentage toward the 86% solvent saving level, as the higher fresh solvent use for the first cycle is outweighed by the more efficient recycled solvent usage in later cycles. Similar to Example 4, the reduction in resin swelling in IPAC results in more solvent drained than charged during the wash sequence.

TABLE 17
Example 5 wash data

| | Resin | Resin | Wash1 | | Wash2 | | Wash3 | | Wash4 | |
|---|---|---|---|---|---|---|---|---|---|---|
| Cycle | mmol | (g) | Charge | Drain | Charge | Drain | Charge | Drain | Charge | Drain |
| Gln | 99.79 | 66.53 | 665 | 749 | 669 | 698 | 668 | 654 | 667 | 666 |
| Ala | 99.38 | 66.25 | 749 | 896 | 698 | 740 | 654 | 665 | 666 | 680 |
| LysSc | 99.01 | 66.01 | 740 | 860 | 665 | 763 | 680 | 693 | 653 | 662 |
| Lys | 98.66 | 65.77 | 763 | 918 | 693 | 753 | 662 | 671 | 675 | 717 |
| Asp | 98.37 | 65.58 | 753 | 883 | 671 | 742 | 717 | 701 | 648 | 708 |
| Leu | 98.12 | 65.41 | 742 | 873 | 701 | 768 | 708 | 722 | 696 | 730 |
| aMeL | 92.96 | 61.97 | 768 | 870 | 722 | 798 | 730 | 743 | 687 | 723 |
| Ile | 92.60 | 61.73 | 798 | 922 | 743 | 800 | 723 | 749 | 771 | 782 |
| Ser | 92.26 | 61.50 | 800 | 895 | 749 | 861 | 782 | 790 | 823 | 876 |
| Tyr | 92.00 | 61.33 | 861 | 930 | 790 | 895 | 876 | 866 | 814 | 876 |
| Asp | 70.41 | 46.94 | 716 | 761 | 693 | 774 | 701 | 725 | 606 | 644 |
| Ser | 70.17 | 46.78 | 774 | 833 | 725 | 791 | 644 | 659 | 649 | 676 |
| Thr | 69.93 | 46.62 | 791 | 814 | 659 | 755 | 676 | 682 | 622 | 652 |
| Phe | 69.70 | 46.47 | 755 | 801 | 682 | 734 | 652 | 665 | 647 | 669 |
| Thr | 69.48 | 46.32 | 734 | 799 | 665 | 703 | 669 | 687 | 593 | 612 |

TABLE 18
Example 5 wash data continued

| | Resin | Resin | Wash5 | | Wash6 | | Wash7 | | Wash8 | |
|---|---|---|---|---|---|---|---|---|---|---|
| Cycle | mmol | (g) | Charge | Drain | Charge | Drain | Charge | Drain | Charge | Drain |
| Gln | 99.79 | 66.53 | 665 | 658 | 669 | 672 | 667 | 662 | 668 | 668 |
| Ala | 99.38 | 66.25 | 658 | 653 | 672 | 674 | 662 | 657 | 668 | 684 |
| LysSc | 99.01 | 66.01 | 674 | 675 | 657 | 661 | 684 | 699 | 678 | 673 |
| Lys | 98.66 | 65.77 | 661 | 648 | 699 | 712 | 673 | 681 | 776 | 791 |
| Asp | 98.37 | 65.58 | 712 | 696 | 681 | 690 | 791 | 777 | 776 | 796 |
| Leu | 98.12 | 65.41 | 690 | 687 | 777 | 789 | 796 | 813 | 777 | 786 |
| aMeL | 92.96 | 61.97 | 789 | 771 | 813 | 830 | 786 | 801 | 728 | 736 |
| Ile | 92.60 | 61.73 | 830 | 823 | 801 | 814 | 736 | 741 | 733 | 740 |
| Ser | 92.26 | 61.50 | 814 | 814 | 741 | 757 | 740 | 753 | 729 | 728 |
| Tyr | 92.00 | 61.33 | 757 | 758 | 753 | 770 | 728 | 743 | 772 | 782 |
| Asp | 70.41 | 46.94 | 616 | 649 | 594 | 624 | 626 | 633 | 560 | 564 |
| Ser | 70.17 | 46.78 | 624 | 622 | 633 | 651 | 564 | 585 | 606 | 619 |
| Thr | 69.93 | 46.62 | 651 | 647 | 585 | 597 | 619 | 625 | 610 | 619 |
| Phe | 69.70 | 46.47 | 597 | 593 | 625 | 641 | 619 | 635 | 603 | 618 |
| Thr | 69.48 | 46.32 | 641 | 636 | 635 | 649 | 618 | 626 | 600 | 619 |

TABLE 19
Example 5 wash data totals

| | Resin | Resin | Total | Total |
|---|---|---|---|---|
| Cycle | mmol | (g) | Charge | Drain |
| Gln | 99.79 | 66.53 | 5337 | 5427 |
| Ala | 99.38 | 66.25 | 5427 | 5650 |
| LysSc | 99.01 | 66.01 | 5432 | 5686 |
| Lys | 98.66 | 65.77 | 5602 | 5890 |
| Asp | 98.37 | 65.58 | 5748 | 5994 |
| Leu | 98.12 | 65.41 | 5887 | 6167 |
| aMeL | 92.96 | 61.97 | 6023 | 6271 |
| Ile | 92.60 | 61.73 | 6134 | 6371 |
| Ser | 92.26 | 61.50 | 6178 | 6474 |
| Tyr | 92.00 | 61.33 | 6351 | 6620 |
| Asp | 70.41 | 46.94 | 5112 | 5375 |
| Ser | 70.17 | 46.78 | 5220 | 5437 |
| Thr | 69.93 | 46.62 | 5214 | 5393 |
| Phe | 69.70 | 46.47 | 5182 | 5356 |
| Thr | 69.48 | 46.32 | 5155 | 5330 |

Upon completion of the synthesis, the synthesized peptide was analyzed for quality using UHPLC-UV (peak area percentage). The overall purity of the peptide measured at 94.4%, well above the process target of 90%. Additionally, the last two washes, wash 7 and wash 8, from three different cycles were analyzed for residual piperidine, shown in Table 20.

TABLE 20
Additional piperidine levels from specific washes

Residual Piperidine (ppm)

| Cycle | Wash 7 | Wash 8 |
|---|---|---|
| aMeL | 2082 | 1303 |
| Tyr | 2794 | 1067 |
| Thr | 2309 | 1039 |

The residual piperidine level after the final wash of 1039 ppm was a bit higher than normal, but the critical level of piperidine has not been determined. In the original process, the average of 9 washes to get to under 500 ppm residual piperidine would likely have needed only 8 washes to get to 1,000 ppm. Using 8 rather than 9 washes for comparison, the IPAC use would still be reduced by 78%. In summary, a 16 mer peptide was synthesized using the solid phase peptide synthesis wash system described herein with IPAC as the wash solvent. The peptide quality was on par with historical data while reducing IPAC use in the post-deprotection wash by 80%.

What is claimed is:

1. A method for washing an amino acid that has been added to a solid phase resin during solid phase peptide synthesis (SPPS), comprising:
adding a first quantity of wash solvent, wherein once the wash with the first quantity of wash solvent is complete, sending the first quantity of wash solvent to waste; and
adding a second quantity of wash solvent, wherein once the wash with the second quantity of wash solvent is complete, sending the second quantity of wash solvent to a container, wherein the second quantity of wash solvent will be used in the first wash for the next amino acid that is added.

2. The method of claim 1, further comprising a third quantity of wash solvent, wherein once the wash with the third quantity of wash solvent is complete, sending the third quantity of wash solvent to a container, wherein the third quantity of wash solvent will be used in the second wash for the next amino acid that is added.

3. The method of claim 2, wherein the process of re-using the solvent in the next amino acid is repeated iteratively, until the last wash is done with fresh solvent.

4. The method of claim 1, wherein the wash solvent is N,N-dimethylformamide (DMF).

5. The method of claim 1, wherein the wash solvent is isopropyl acetate (IPAC).

6. The method of claim 1, wherein a base is present with the amino acid and solid phase resin and the washing reduces the base concentration to the same level as when all fresh wash solvent is used.

7. The method of claim 6, wherein the base concentration is reduced to less than about 1000 ppm.

8. The method of claim 6, wherein the base concentration is reduced to less than about 500 ppm.

9. The method of claim 6, wherein the base is a secondary amine.

10. The method of claim 9, wherein the secondary amine is piperidine.

11. The method of claim 1, wherein total quantity of wash solvent used is decreased by at least about 75% compared to using only fresh wash solvent.

12. The method of claim 1, wherein total quantity of wash solvent used is decreased by at least about 80% compared to using only fresh wash solvent.

13. The method of claim 1, wherein total quantity of wash solvent used is decreased by about 75% compared to using only fresh wash solvent.

14. The method of claim 1, wherein the total quantity of wash solvent used is decreased by about 80% compared to using only fresh wash solvent.

15. The method of claim 1 any of claims 1 to 14, wherein the method steps are controlled automatically.

16. The method of claim 1 any of claims 1 to 15, wherein two or more of the quantities of wash solvent are combined into a single volume and applied to the resin as a plug-flow wash with fractions of wash effluent diverted to the wash receptacle or container.

17. A system for washing an amino acid that has been added to a solid phase resin during solid phase peptide synthesis (SPPS), comprising:
a first container and a waste receptacle; and
a first quantity of wash solvent and a second quantity of wash solvent,
wherein once the wash with the first quantity of wash solvent is complete, the first quantity of wash solvent is sent to the waste receptacle, and
wherein, once the wash with the second quantity of wash solvent is complete, the second quantity of wash solvent is sent to the first container, and
wherein the second quantity of wash solvent will be used in the first wash for the next amino acid that is added.

18. The system of claim 17, further comprising a second container and a third quantity of wash solvent, wherein once the wash with the third quantity of wash solvent is complete, the third quantity of wash solvent is sent to the second container, container, wherein the third quantity of wash solvent will be used in the second wash for the next amino acid that is added.

19. The system of claim 17, wherein a base is present with the amino acid and solid phase resin and the system reduces base concentration to the same level as when all fresh wash solvent is used.

20. The system of claim 17, wherein the system reduces base concentration to less than about 1000 ppm.

21. The system of claim 17, wherein the system reduces base concentration to less than about 500 ppm.

22. The system of claim 17, wherein total volume of wash solvent used is decreased by at least about 75% compared to using only fresh wash solvent.

23. The system of claim 17, wherein total volume of wash solvent used is decreased by at least about 80% compared to using only fresh wash solvent.

24. The system of claim 17, wherein total volume of wash solvent used is decreased by about 75% compared to using only fresh wash solvent.

25. The system of claim 17, wherein the total volume of wash solvent used is decreased by about 80% compared to using only fresh wash solvent.

26. The system of claim 17, wherein the system is an independent add-on system adapted to be connected to an SPPS system.
